(19) United States
(12) Patent Application Publication
(10) Pub. No.: US 2010/0028879 A1
(43) Pub. Date: Feb. 4, 2010

(54) Modulators of HSD17b7 for treating acne or hyperseborrhea

(75) Inventors: Fernand Labrie, Quebec (CA); Michel Rivier, Nice (FR); Van Luu-The, Charny (CA); Ezequiel L. Calvo, Quebec (CA); Jerome Aubert, Grasse (FR); Sophie Deret, Mougins (FR); Johannes Voegel, Chateauneut/Grasse (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, Biot (FR)

(21) Appl. No.: 12/320,169

(22) Filed: Jan. 21, 2009

Related U.S. Application Data
(63) Continuation of application No. PCT/FR2007/051683, filed on Jul. 18, 2007.

(30) Foreign Application Priority Data
Jul. 19, 2006 (FR) 0653029

(51) Int. Cl. C12Q 1/68; G01N 33/573

(52) U.S. Cl. 435/6; 435/7.4

(57) ABSTRACT

An in vitro method for screening candidate compounds for the preventive or curative treatment of acne, includes the determination of the capacity of a compound to modulate the expression or the activity of hydroxysteroid (17-beta) dehydrogenase type 7, and the use of modulators of the expression or activity of this enzyme for the treatment of acne or skin disorders associated with a hyperseborrhea; methods for the in vitro diagnosis or in vitro prognosis of these pathologies are also described.

### CROSS-REFERENCE TO PRIORITY/PCT APPLICATIONS

This application claims priority under 35 U.S.C. §119 of FR 0653029, filed Jul. 19, 2006, and is a continuation/national phase of PCT/FR 2007/051683, filed Jul. 18, 2007, and designating the United States (published in the French language on Jan. 24, 2008 as WO 2008/009856 A2; the title and abstract were also published in English), each hereby expressly incorporated by reference in its entirety and each assigned to the assignee hereof.

### BACKGROUND OF THE INVENTION

1. Technical Field of the Invention

The present invention relates to the identification and administration of hydroxysteroid (17-beta) dehydrogenase 7 (HSD17b7) modulating compounds for the treatment of acne and skin disorders associated with a hyperseborrhea. This invention also relates to methods for the in vitro diagnosis or in vitro prognosis of these pathologies.

2. Description of Background and/or Related and/or Prior Art

A hyperseborrheic greasy skin is characterized by excessive secretion and excretion of sebum. Conventionally, a sebum level greater than 200 μg/cm², measured in the region of the forehead, is considered as being characteristic of a greasy skin. A greasy skin is often associated with a desquamation defect, a glistening complexion and a thick skin grain. In addition to these aesthetic disorders, excess sebum can serve as a support for the anarchical development of saprophytic bacterial flora (P. acnes in particular), and cause the appearance of comedones and/or acne lesions.

This stimulation of the production of sebaceous glands is induced by androgens. Acne is in fact a chronic disease of the pilosebaceous follicle under hormonal control. A hormone therapy against acne is one possibility of treatment for women, the aim being to prevent the effects of androgens on the sebaceous gland. In this context, use is generally made of oestrogens, anti-androgens or agents reducing the production of androgens by the ovaries or the adrenal gland. The anti-androgens administered for the treatment of acne include in particular spironolactone, cyproterone acetate and flutamide. However, these agents have severe side effects. Thus, any pregnancy must be absolutely prevented, in particular because of a risk of feminization for the male foetus. These agents are banned in male patients.

Need therefore exists to identify mediators downstream of the action of the steroid hormones and to modulate them in order to provide a similar therapeutic profile, but with reduced side effects.

### SUMMARY OF THE INVENTION

It has now been discovered that the gene HSD17b7 was expressed in the human sebaceous glands, and that its expression was regulated by androgens, in vitro, in a mouse preputial gland model. Thus, targeting this gene, or its expression product the enzyme hydroxysteroid (17-beta)dehydrogenase 7 is now proposed to prevent or improve acne phenomena and skin disorders associated with a hyperseborrhea, in particular the appearance of greasy skin.

The expression acne is understood to mean all the forms of acne, namely, in particular acne vulgaris, comedo type acne, polymorphic acne, nodulocystic acne, acne conglobata, or secondary acnes such as solar acne, acne medicamentosa or occupational acne.

This invention also provides in vitro diagnostic or in vitro prognostic methods based on the detection of the expression or activity of hydroxysteroid (17-beta)dehydrogenase type 7.

### BRIEF DESCRIPTION OF THE DRAWINGS

FIGS. 1A and 1B are graphs which show the measurement of the expression of the HSD17B7 gene in gonadectomized male mice treated with various vehicles,

FIGS. 2A and 2B are graphs presenting a kinetic study of 15 minutes to 96 hours using two different probe sets which hybridize over different regions of the HSD17B7 gene, and

FIGS. 2C and 2D are graphs presenting a kinetic study of 1 hour to 24 hours using two different probe sets which hybridize over different regions of the HSD17B7 gene.

### DETAILED DESCRIPTION OF BEST MODE AND SPECIFIC/PREFERRED EMBODIMENTS OF THE INVENTION

HSD17B7:

The protein HSD17b7, which denotes hydroxysteroid (17-beta) dehydrogenase type 7, belongs to the family of hydroxysteroid (17-beta) dehydrogenases which catalyze the last reaction of the biosynthesis of several oestrogens and androgens, such as oestradiol, 5α-androstane-3β,17β-diol, testosterone, and dihydrotestosterone. Twelve isoforms are known which exhibit specific reducing or oxidizing activities (Poirier, Current Medicinal Chemistry, 2003, 10:453-477; Thiboutot et al, J. Invest Dermatol., 1998, 111: 390-395).

Since an androgenic stimulation is responsible for an increase in the production of sebum, which can induce acne lesions, the inhibition of 17beta-hydroxysteroid dehydrogenases, key enzymes in the metabolism of androgens, has been indicated as a potential treatment for acne. In these cases, a non-specific inhibition of one or more hydroxysteroid (17-beta) dehydrogenases is sought. Members of the HSD17b family have less than 30% homology in primary structure with each other. The various types of HSD17b differ in their substrate and their specificities for cofactors (Labrie et al. (2000) Trends Endocrinol Metab., 11:421-7).

In addition, certain types of HSD17b are involved in the pathogenicity of certain human disorders. HSD17b-3 is known to be involved in the development of pseudohermaphrodism, HSD17b-8 plays a role in polycystic kidney disease and HSD17b-4 is linked to the onset of bifunctional enzyme deficiency. Through the modulation of a particular HSD17b, it is possible to influence or control the local and paracrine concentration of oestrogens and androgens in various tissues. Several reversible and irreversible inhibitors of HSD17b-2 enzymes of steroidal or non-steroidal origin are already known in the literature (Poirier D. (2003) Curr Med Chem., 10:453-77), such as for example WO 02/26706 which describes inhibitors of HSD17b-2 of non-steroidal origin. JP48042271 describes compounds useful as anti-inflammatory compounds. U.S. Pat. No. 5,597,823 describes, for its part, adrenergic antagonists useful for the treatment of benign prostatic hyperplasia. JP62132884 discloses benzylthienopyrimidinones for the treatment of cardiovascular disorders.

WO 2005/032527 relates to the use of thiophenepyrimidinone derivatives for the treatment or prevention of steroid hormone-dependent diseases by the inhibition of 17β-HSD-1. Since each HSD17b subtype has a selective affinity for a substrate and a specific tissue distribution, the selectivity of action may be achieved by targeting a specific isozyme of HSD17b.

In addition to its capacity to synthesize 17β-oestradiol in vitro for which there is no proof of physiological relevance to date, the protein HSD17b7 also exhibits a 3-ketosteroid reductase activity, which is the aspect of interest in the present invention: HSD17b7 is capable of complementing a deficiency in the 3-ketosteroid reductase activity in Erg27p-deficient yeasts and thus allows the growth of cells in a sterol-free medium. HSD17b7 is located in the endoplasmic reticulum, which is the site for the post-squalene part of the synthesis of cholesterol, and is specifically expressed in the tissues involved in congenital cholesterol deficiency disorders (Marijanovic et al, Molecular Endocrinology 2003, 17(9):1715-1725).

It is therefore this activity of the enzyme in the biosynthesis of cholesterol which is of interest in the present invention, and the selective modulation of this enzyme.

In the context of the invention, the term “HSD17b7 gene” or “HSD17b7 nucleic acid” means the gene or nucleic acid sequence which encodes hydroxysteroid (17-beta)dehydrogenase type 7. If the intended target is preferably the human gene or its expression product, the invention may also call into play cells expressing a heterologous hydroxysteroid (17-beta)dehydrogenase type 7, through genomic integration or transient expression of an exogenous nucleic acid encoding the enzyme (for example, an enzyme from another organism).

A human cDNA sequence for HSD17b is reproduced in the annex (SEQ ID No. 1). It is the sequence NM016371 whose coding part is located from nucleic acid 96 to 1121.

Diagnostic Applications:

The present invention also features an in vitro method for the diagnosis or monitoring of the progression of acne lesions or of a skin disorder associated with a hyperseborrhea in a subject, comprising comparing the expression or the activity of the hydroxysteroid (17-beta) dehydrogenase type 7 (HSD17B7), the expression of its gene or the activity of at least one of its promoters, in a biological sample from a subject compared with a biological sample from a control subject.

The expression of the protein may be determined by an assay of the protein (HSD17B7)by radioimmunoassay, for example by ELISA assay. Another method, in particular for measuring the expression of the gene, is to measure the quantity of corresponding mRNA, by any method as described above. An assay of the activity of the enzyme may also be employed.

In the context of a diagnosis, the “control” subject is a “healthy” subject.

In the context of a monitoring of the progression of acne lesions or of a skin disorder linked to a hyperseborrhea, the “control subject” refers to the same subject at a different time, which preferably corresponds to the start of the treatment (To). This measurement of the difference in the expression or activity of the protein, or the expression of its gene or the activity of at least one of its promoters, makes it possible in particular to monitor the efficacy of a treatment, in particular a treatment with an enzyme modulator, as indicated above or with another treatment against acne or a skin disorder associated with a hyperseborrhea. Such a monitoring can reassure the patient regarding the justification or the need for pursuing this treatment.

The present invention also features an in vitro method for determining the predisposition of a subject to develop acne lesions or a skin disorder associated with a hyperseborrhea, comprising comparing the expression or the activity of the protein HSD17b7, the expression of its gene or the activity of at least one of its promoters, in a biological sample from a subject compared with a biological sample from a control subject.

Here again, the expression of the HSD17b7 protein may be determined by an assay of this protein by radioimmunoassay, for example by ELISA assay. Another method, in particular for measuring the expression of its gene, is to measure the quantity of corresponding mRNA by any method as described above. An assay of the activity of the enzyme may also be employed.

The subject tested is here an asymptomatic subject with no skin disorder linked to a hyperseborrhea or an acne. The “control” subject in this method means a “healthy” reference subject or population. The detection of this predisposition allows the putting in place of a preventive treatment and/or an increased monitoring of the signs linked to acne or to a skin disorder associated with a hyperseborrhea.

In these in vitro diagnostic or prognostic methods, the biological test sample may be any biological fluid sample or a sample of a biopsy. Preferably, the sample may be a preparation of skin cells obtained for example by desquamation or biopsy. It may also be sebum.

Screening Methods:

This invention also features an in vitro method for screening candidate compounds for the preventive and/or curative treatment of acne, or for the skin disorders associated with a hyperseborrhea, comprising determining the capacity of a compound to modulate the expression or activity of hydroxysteroid (17-beta)dehydrogenase type 7 or the expression of its gene or the activity of at least one of its promoters, a modulation of the expression or of the activity of the enzyme indicating the usefulness of the compound for the preventive or curative treatment of acne or of the skin disorders associated with a hyperseborrhea. The method therefore makes it possible to select the compounds capable of modulating the expression or activity of HSD17b7, or the expression of its gene or the activity of at least one of its promoters.

More particularly, this invention features an in vitro method for screening candidate compounds for the preventive and/or curative treatment of acne and/or skin disorders associated with a hyperseborrhea, comprising the following steps:

a. preparing at least two biological samples or reaction mixtures;

b. bringing one of the samples or reaction mixtures into contact with one or more test compounds;

c. measuring the expression or activity of the protein hydroxysteroid (17-beta) dehydrogenase type 7, the expression of its gene or the activity of at least one of its promoters,

d. selecting the compounds for which a modulation of the expression or activity of the protein hydroxysteroid (17-beta) dehydrogenase type 7, or a modulation of the expression of its gene or a modulation of the activity of at least one of its promoters, is measured in the sample or mixture treated in b), compared with the untreated sample or mixture.

The expression “modulation” is understood to mean any effect on the expression or activity of the enzyme, the expression of the gene or the activity of at least one of its promoters, namely, optionally a partial or complete stimulation, but preferably a partial or complete inhibition. The difference in expression obtained with the test compound compared with a control prepared in the absence of the compound is significant from 25% or more.

In the present text, unless otherwise specified, “expression of a protein” is understood to mean the quantity of this protein.

The expression “activity of a protein” is understood to mean its biological activity.

The expression “activity of a promoter” is understood to mean the capacity of this promoter to trigger the transcription of the DNA sequence coded downstream of this promoter (and therefore indirectly the synthesis of the corresponding protein).

The test compounds may be of any type. They may be of a natural origin or may have been produced by chemical synthesis. This may be a library of structurally defined chemical compounds, non-characterized compounds or substances or a mixture of compounds.

Various techniques may be used to test these compounds and identify the compounds of therapeutic interest, modulators of the expression or the activity of hydroxysteroid (17-beta) dehydrogenase type 7.

According to a first embodiment, the biological samples are cells transfected with a reporter gene that is operably linked to all or part of the promoter of the HSD17b7 gene, and step c) described above entails measuring the expression of the said reporter gene.

The reporter gene may in particular encode an enzyme which, in the presence of a given substrate, leads to the formation of colored products, such as CAT (chloramphenicol acetyltransferase), GAL (beta-galactosidase) or GUS (beta-glucuronidase). This may also be the luciferase gene or GFP (Green Fluorescent Protein). The assay of the protein encoded by the reporter gene, or its activity, is carried out in a conventional manner by calorimetric, fluorometric or chemiluminescent techniques, among others.

According to a second embodiment, the biological samples are cells expressing the HSD17b7 gene encoding hydroxysteroid (17-beta) dehydrogenase type 7, and step c) described above entails measuring the expression of the said gene.

The cell employed here may be of any type. This may be a cell endogenously expressing the HSD17b7 gene, such as, for example, a liver cell, an ovarian cell or even better a sebocyte. It is also possible to employ organs of human or animal origin, such as for example the preputial gland, clitorial gland or sebaceous gland of the skin.

This may also be a cell transformed with a heterologous nucleic acid encoding a hydroxysteroid (17-beta) dehydrogenase 7, preferably of human origin, or of mammalian origin.

A wide variety of host cell systems may be employed, such as, for example, Cos-7, CHO, BHK, 3T3, HEK293 cells. The nucleic acid may be stably or transiently transfected by any method known to one skilled in this art, for example using calcium phosphate, DEAE-dextran, liposome, viruses, electroporation or microinjection.

In these methods, the level of expression of the HSD17B7 gene may be determined by evaluating the level of transcription of the said gene, or its level of translation.

The expression level of transcription of a gene is understood to mean the quantity of corresponding mRNA produced. The expression level of translation of a gene is understood to mean the quantity of corresponding protein produced.

One skilled in this art is familiar with techniques allowing the quantitative or semi-quantitative detection of the mRNA of a gene of interest. The techniques based on the hybridization of mRNA with specific nucleotide probes are the most common (Northern Blot, RT-PCR, protection using RNase). It may be advantageous to employ detection markers such as fluorescent, radioactive or enzymatic agents or other ligands (for example avidin/biotin).

In particular, the expression of the gene may be measured by real-time PCR or by protection using RNase. The expression protection using RNase is understood to mean the detection of a known mRNA among poly(A) RNAs of a tissue, which may be carried out with the aid of a specific hybridization with a labeled probe. The probe is a labeled (radioactive) complementary RNA for the messenger to be detected. It may be constructed from a known mRNA whose cDNA, after RT-PCR, has been cloned into a phage. The poly(A) RNA of the tissue where the sequence is to be detected is incubated with this probe under slow hybridization conditions in liquid medium. RNA:RNA hybrids are formed between the mRNA to be detected and the anti-sense probe. The hybridized medium is then incubated with a mixture of ribonucleases specific for single-stranded RNA, such that only the hybrids formed with the probe can withstand this digestion. The product of digestion is then deproteinized and repurified before being analyzed by electrophoresis. The labeled hybridized RNAs are detected by autoradiography.

The level of translation of the gene is evaluated for example by immunological assay of the product of the said gene. The antibodies employed for this effect may be of the polyclonal or monoclonal type. Their production involves conventional techniques. An anti-hydroxysteroid (17-beta) dehydrogenase 7 polyclonal antibody may, inter alia, be obtained by immunization of an animal such as a rabbit or a mouse, with the whole enzyme, collection and then depletion of the anti-serum according to methods known per se by persons skilled in the art. A monoclonal antibody may, inter alla, be obtained by the conventional Kôhler and Milstein method (Nature (London), 256: 495-497 (1975)). Other methods of preparation of monoclonal antibodies are also known. It is possible, for example, to produce monoclonal antibodies by expressing a nucleic acid cloned from a hybridoma. It is also possible to produce antibodies by the phage display technique by introducing antibody cDNAs into vectors, which are typically filamentous phages which display V gene libraries at the surface of the phage (for example, fUSE5 for E. coli).

The immunological assay may be carried out in a solid phase or in a homogeneous phase; in a single stage or in two stages; as a sandwich method or as a competitive method, by way of non-limiting examples. According to a preferred embodiment, the capture antibody is immobilized on a solid phase. It is possible to use, by way of non-limiting examples of a solid phase, microplates, in particular polystyrene microplates, or solid particles or beads, paramagnetic beads.

ELISA assays, radio-immunoassays or any other detection technique may be carried out in order to reveal the presence of the antigen-antibody complexes formed.

The characterization of the antigen-antibody complexes, and more generally of the isolated or purified proteins, but also recombinant proteins (obtained in vitro and in vitro), may be carried out by mass spectrometry analysis. This identification is made possible by virtue of the analysis (determination of the mass) of peptides generated by the enzymatic hydrolysis of the proteins (trypsin in general). Generally, the proteins are isolated according to methods known to one skilled in this art, prior to the enzymatic digestion. The analysis of the peptides (in hydrolysate form) is performed by separation of the peptides by HPLC (nano-HPLC) based on their physicochemical properties (reversed phase). The determination of the mass of the peptides thus separated is carried out by ionization of the peptides or by direct coupling to mass spectrometry (electrospray ESI mode), or after deposition and crystallization in the presence of a matrix known to one skilled in this art (analysis in MALDI mode). The proteins are then identified using appropriate software (for example Mascot).

According to a third embodiment, step a) described above entails preparing reaction mixtures comprising an enzyme hydroxysteroid (17-beta) dehydrogenase 7 and a substrate of the enzyme, and step c) described above entails measuring the enzyme activity. Preferably, step a) entails preparing reaction mixtures comprising an enzyme hydroxysteroid (17-beta)dehydrogenase 7 and a substrate of the enzyme, and a reductase system, and step c) entails measuring the enzyme activity.

The enzyme may be produced according to customary techniques using Cos-7, CHO, BHK, 3T3 and HEK293 cells. It may also be produced with the aid of microorganisms such as bacteria (for example E. coli or B. subtilis), yeasts (for example Saccharomyces, Pichia) or insect cells, such as Sf9 or Sf21.

An example of measurement of the enzyme activity may be performed according to the conventional method of Marijanovic et al., Mol Endocrinol, September 2003, 17(9):1715-1725. It entails measuring the conversion of zymosterone to zymosterol by hydroxysteroid (17-beta) dehydrogenase. The enzyme is incubated for 90 min at 37° C. in a reaction medium containing 880 μL of buffer (10 mM KPl, 0.05% BSA, and 1 mM EDTA pH 8), 100 μL of bacterial lysate, 10 μL of NADPH (5 mg/ml), 10 μL of substrate (zymosterone, or 17β-oestradiol, or androsterone). The steroids produced during the reaction are extracted by reverse-phase chromatography in an RP18 column and eluted twice with a methanol:chloroform 1:1 solution and once with chloroform alone. After evaporation of the solvent, the steroids are solubilized in chloroform, separated by thin-layer chromatography (Merck) using a toluene:ethyl acetate 80:20 solution as mobile phase and detected by spraying a 30% H₂SO₄ solution in ethanol and developed at 135° C. The identification of the substrates and the metabolites is carried out by comparison with the reference steroids.

Modulators of the Enzyme:

The present invention also features the use of a modulator of the human enzyme hydroxysteroid (17-beta) dehydrogenase 7 which can be obtained according to one of the above methods for the preparation of a medicament intended for the preventive and/or curative treatment of acne, or of skin disorders associated with a hyperseborrhea.

A method for the preventive and/or curative treatment of acne, or of skin disorders associated with a hyperseborrhea, is thus described here, the regime or regimen comprising the administration of a therapeutically effective quantity of a modulator of the human enzyme hydroxysteroid (17-beta) dehydrogenase 7, to a patient requiring such a treatment.

This invention also features the cosmetic application of a modulator of the human enzyme hydroxysteroid (17-beta) dehydrogenase 7 for the aesthetic treatment of greasy skins.

Preferably, the modulator is an inhibitor of the enzyme. The term “inhibitor” refers to a chemical compound or substance which substantially eliminates or reduces the enzymatic activity of hydroxysteroid (17-beta) dehydrogenase 7. The term “substantially” means a reduction of at least 25%, preferably of at least 35%, preferably still of at least 50%, and more preferably of at least 70% or 90%. More particularly, it may be a compound which interacts with, and blocks, the catalytic site of the enzyme, such as compounds of the competitive inhibitor type.

A preferred inhibitor interacts with the enzyme in solution at inhibitor concentrations of less than 1 μM, preferably of less than 0.1 μM, preferably still of less than 0.01 μM.

The modulator compound may be an anti-hydroxysteroid (17-beta) dehydrogenase 7 inhibitory antibody, preferably a monoclonal antibody. Advantageously, such an inhibitory antibody is administered in a quantity sufficient to obtain a plasma concentration of about 0.01 μg per ml to about 100 μg/ml, preferably of about 1 μg per ml to about 5 μg/ml.

The modulator compound may also be a polypeptide, a DNA or RNA anti-sense polynucleotide, an si-RNA or a PNA (“peptide nucleic acid”, polypeptide chain substituted with purine and pyrimidine bases whose spatial structure mimics that of DNA and allows hybridization thereto).

Several hydroxysteroid (17-beta) dehydrogenase inhibitors are known, but the invention preferably aims at the use of inhibitors specific for isoform 7. The invention comprises the administration of such hydroxysteroid (17-beta) dehydrogenase 7 inhibiting compounds for the preventive and/or curative treatment of acne or skin disorders associated with a hyperseborrhea.

The modulator compounds are formulated in pharmaceutical compositions, in combination with a pharmaceutically acceptable vehicle. These compositions may be administered for example orally, parenterally or topically. Preferably, the pharmaceutical composition is applied topically. By the oral route, the pharmaceutical composition may be provided in the form of tablets, gelatin capsules, sugar-coated tablets, syrups, suspensions, solutions, powders, granules, emulsions, suspensions of microspheres or nanospheres or lipid or polymer vesicles allowing controlled release. By the parenteral route, the pharmaceutical composition may be provided in the form of solutions or suspensions for infusion or injection.

By the topical route, the pharmaceutical composition is more particularly useful for the treatment of the skin and the mucous membranes and may be provided in the form of salves, creams, milks, ointments, powders, impregnated pads, solutions, gels, sprays, lotions or suspensions. It may also be provided in the form of suspensions of microspheres or nanospheres or of lipid or polymer vesicles or of polymer patches or hydrogels allowing controlled release. This composition for topical application may be provided in anhydrous form, in aqueous form or in the form of an emulsion. In a preferred embodiment, the pharmaceutical composition is provided in the form of a gel, a cream or a lotion.

The composition may comprise an amount of HSD17b7 modulator ranging from 0.001 to 10% by weight, in particular from 0.01 to 5% by weight relative to the total weight of the composition.

The pharmaceutical composition may additionally contain inert additives or combinations of these additives, such as:

wetting agents;

taste enhancing agents;

preservatives such as para-hydroxybenzoic acid esters;

stabilizing agents;

moisture regulating agents;

pH regulating agents;

osmotic pressure modifying agents;

emulsifying agents;

UV-A and UV-B screening agents;

and antioxidants, such as alpha-tocopherol, butylated hydroxyanisole or butylated hydroxytoluene, Super Oxide Dismutase, Ubiquinol or certain metal chelators.

Legends for the Figures:

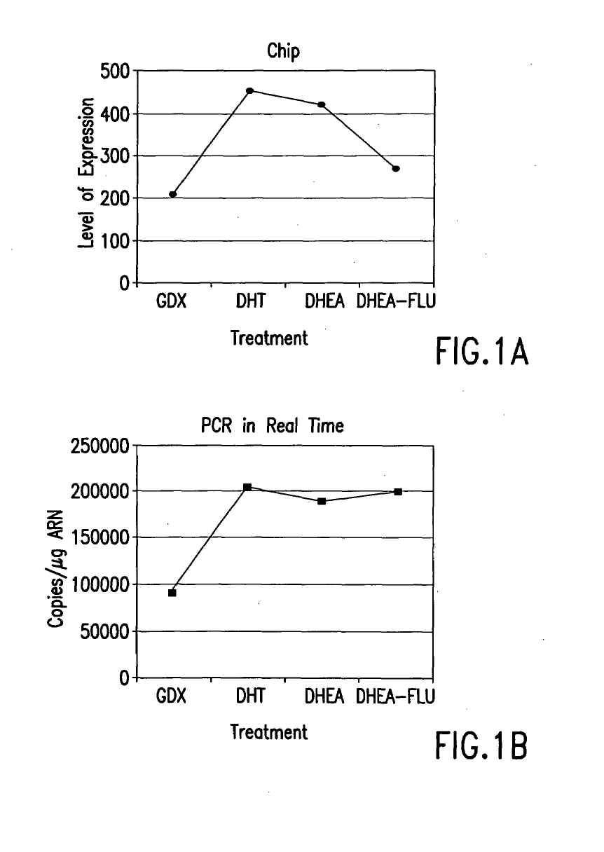

FIGS. 1A and 1B are graphs which show the measurement of the expression of the HSD17B7 gene in gonadectomized male mice treated with the vehicle, DHT, DHEA or the combination of DHEA-Flutamide for a period of 7 days once per day (long-term treatment). The results obtained by the Affymetrix technique (FIG. 1A) were confirmed by the real-time RT-PCR technique (FIG. 1B).

GDX: gonadectomized mice treated with the vehicle.

DHT: gonadectomized mice treated with Dihydrotestosterone (agonist of the androgen receptor).

DHEA: gonadectomized mice treated with Dihydroepiandrosterone (precursor of the steroid hormones; in the preputial glands metabolized to the active androgen).

DHEA-Flu: gonadectomized mice treated with a combination of Dihydroepiandrosterone and Flutamide (antagonists of the androgen receptor; which block the effects of the DHT and DHEA agonists).

Level of expression: level of expression of the mRNA.

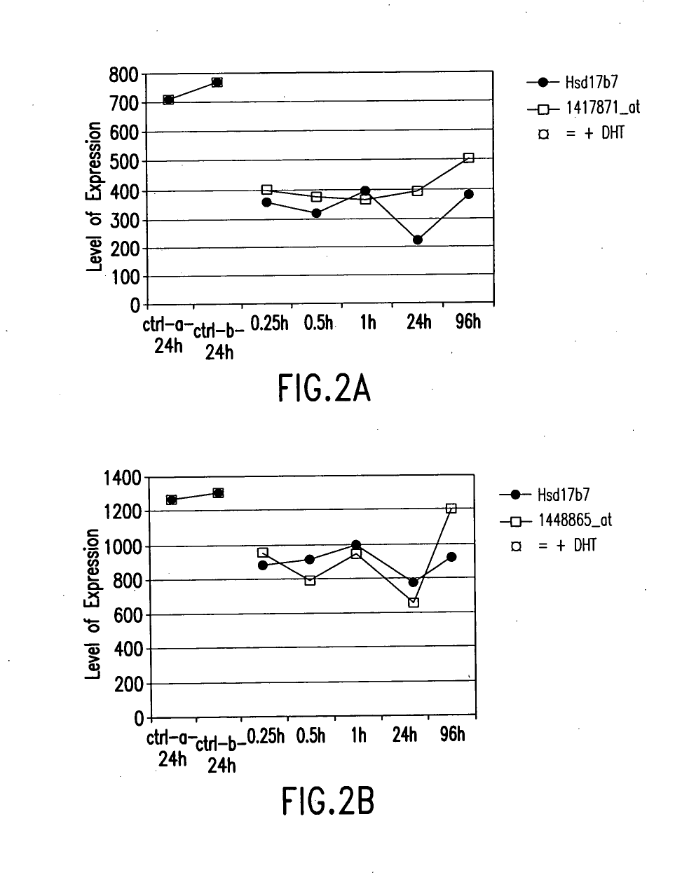

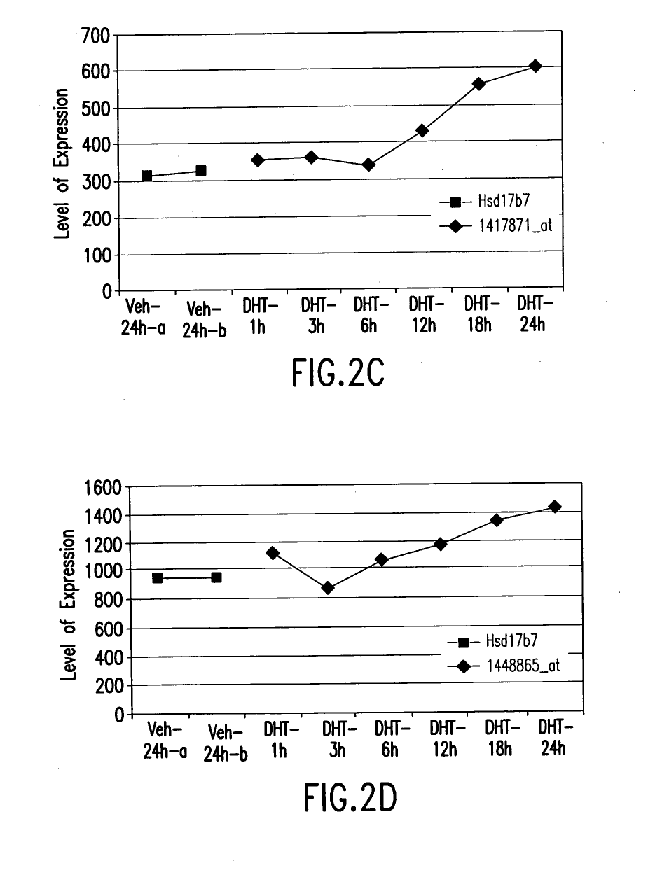

FIGS. 2A and 2B are graphs presenting a kinetic study of 15 minutes to 96 hours using two different probe sets which hybridize over different regions of the HSD17B7 gene. With respect to FIG. 2A, the probe set used is 1417871_at, for FIG. 2B, the probe set used is 1448865_at. The various observation times are identical in both experiments.

In FIG. 2A, points ctrl-a-24h and ctrl-b-24 show the level of expression of HSD17B7 of control mice (=non-gonadectomized mice; duplicate) at the 24 hour point. The next points are from gonadectomized mice and indicate the successive times (in hours) of the kinetic study.

Level of expression: level of expression of mRNA.

Square: expression in the gonadectomized mice following treatment with DHT at the time zero.

Circle: expression in gonadectomized mice without DHT treatment at the time zero.

FIGS. 2C and 2D are graphs presenting a kinetic study of 1 hour to 24 hours using two different probe sets which hybridize over different regions of the HSD17B7 gene. FIG. 2C represents the level of expression of HSD17B7 using the probe set 1417871_at, FIG. 2D represents the level of expression of HSD17B7 using the probe set 1448865_at. The points Veh-24h-a and Veh-24h-b show the level of expression of HSD17B7 of gonadectomized mice untreated with DHT.

Level of expression: level of expression of the mRNA.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Examples

### Experimental Data

### Example 1

### Expression of HSD17B7 in the Human Sebaceous Gland and in the Human Epidermis

Human sebaceous glands were separated from the human epidermis by treatment with dispase and microdissection under a binocular lens. Samples of total RNA were prepared from the sebaceous glands and from the epidermis.

The expression of the genes was analyzed on an Affymetrix station (microfluidic model; hybridization oven; scanner; computer) following the protocols provided by the company. Briefly, the total RNA isolated from the tissues was transcribed to cDNA. From the double-stranded cDNA, a cRNA labeled with biotin was synthesized using T7 polymerase and a precursor NTP conjugated to biotin. The cRNAs were then fragmented to small sized fragments. All the molecular biology steps are checked using the Agilent “Lab on a chip” system in order to confirm the good efficiencies of the enzymatic reactions. The Affymetrix chip is hybridized with the biotinylated cRNA, rinsed and then fluorescence labeled using a fluorophore conjugated to streptavidin. After washings, the chip is scanned and the results are calculated using the MAS5 software provided by Affymetrix. An expression value is obtained for each gene as well as the indication of the significance of the value obtained. The calculation of the significance of the expression is based on the analysis of the signals, which are obtained following hybridization of the cRNA of a given gene with an oligonucleotide that is a perfect match compared with an oligonucleotide which contains a single mismatch in the central region of the oligonucleotide (see Table 1).

TABLE 1
Measurement of the expression of HSD17B7 in the epidermis and in
the sebaceous gland through use of the Affymetrix technology.

| | | | | Significance of | Significance of |
|---|---|---|---|---|---|
| | | Expression | Expression | the expression* | the expression* |
| Affymetrix | Name of | in the human | in the human | in the human | in the human |
| identifier | the gene | sebaceous gland | epidermis | sebaceous gland | epidermis |
| 220081_x_at | HSD17B7 | 155 | 111 | 1 | 1 |

*Indicator of the significance of the expression of the gene analyzed in the sample indicated: presence (=1) or absence (=0).

Results:

HSD17B7 is well expressed in both tissues (sebaceous gland, epidermis). Differential analysis between the expression in the human sebaceous gland and the human epidermis shows that the slightly higher expression in the sebaceous gland is not significant compared with the value observed in the epidermis.

### Example 2

### Expression of HSD17B7 in the Mouse Preputial Gland

The mouse preputial glands show differentiation of the sebocyte type and are used as an experimental model for a sebaceous gland. They have a sufficient size to allow isolation of RNA without having recourse to microdissection technologies.

The expression of HSD17B7 in the mouse preputial glands has been analyzed under conditions of deficiency of steroid hormones (in particular of androgenic hormones) following a gonadectomy. The gonadectomized animals were then treated with physiological quantities of Dihydrotestosterone (DHT) or Dihydroepiandrosterone (DHEA) in order to restore a physiological level of androgenic hormones, or as a control experiment with a DHEA-Flutamide combination in which the Flutamide, an antagonist of the androgen receptors, blocks the effect of DHEA. Comparison of the gene expression under these experimental conditions makes it possible to unambiguously identify the modulation or non-modulation of the gene expression of a gene in question by the androgenic hormones.

The gene expression was analyzed using the Affymetrix technology described above (FIG. 1A) and the results were then confirmed by the real-time PCR technique (FIG. 1B).

The real-time PCR was carried out using the protocols provided by the company Applied Biosystems using the 7900HT Sequence Detection System. The total RNA isolated from the tissues is transcribed (RT) to cDNA and the latter is amplified by PCR (polymerase chain reaction). The progress of the PCR is monitored in real time using fluorescent TaqMan probes which allow precise quantification of the quantity of mRNA of a given gene present in the biological sample at the start.

Result:

The mRNA for HSD17B7 is induced by a chronic treatment for 7 days with androgens in the preputial gland.

B. Male mice were gonadectomized and were then treated with the vehicle or DHT. The preputial glands were removed for a period ranging up to 4 days (androgenic treatment alone—observation of a short-term kinetics). The RNA was isolated and the expression of the genes was analyzed by the Affymetrix technique using two different probe sets: 1417871_at (FIGS. 2A and 2C) and 1448865_at (FIGS. 2B and 2D). FIGS. 2A, 2B, 2C and 2D represent the relative level of expression of the mRNA as a function of time.

Result:

Gonadectomy which causes a steroid hormone deficiency induces a repression of the quantity of mRNA for HSD17B7 in the mouse preputial gland (FIGS. 2A and 2B). The mRNA for HSD17B7 in the mouse preputial gland is moderately induced by a short-term treatment with DHT (effect visible at 24 and 96 hours) (FIGS. 2A and 2B).

The mRNA for HSD17B7 in the mouse preputial gland is induced by a short-term treatment with DHT (effect starting at 12 hours and well visible at 18 and 24 hours) (FIG. 2C and FIG. 2D).

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An in vitro method for screening candidate compounds for the preventive and/or curative treatment of acne, or skin disorders associated with a hyperseborrhea, comprising determining the capacity of a candidate compound to modulate the expression or activity of the protein hydroxysteroid (17-beta) dehydrogenase 7, the expression of its gene or the activity of at least one of its promoters.

2. An in vitro method for screening candidate compounds for the preventive and/or curative treatment of acne or skin disorders associated with a hyperseborrhea as defined by claim 1, comprising the following steps:
a. preparing at least two biological samples or reaction mixtures;
b. bringing one of the samples or reaction mixtures into contact with one or more test compounds;
c. measuring the expression or activity of the protein hydroxysteroid (17-beta) dehydrogenase 7, the expression of its gene or the activity of at least one of its promoters, in biological samples or reaction mixtures;
d. selecting the compounds for which a modulation of the expression or activity of the protein hydroxysteroid (17-beta) dehydrogenase 7, or a modulation of the expression of its gene or a modulation of the activity of at least one of its promoters, is measured in the sample or mixture treated in b), compared with the untreated sample or mixture.

3. The in vitro method as defined by claim 2, wherein the compounds selected in step d) inhibit the expression or the activity of the protein hydroxysteroid (17-beta) dehydrogenase 7, the expression of its gene or the activity of at least one of its promoters.

4. The in vitro method as defined by claim 2, wherein the biological samples are cells transfected with a reporter gene that is operably linked to all or part of the promoter of the HSD17B7 gene, and in that step c) comprises measuring the expression of the said reporter gene.

5. The in vitro method as defined by claim 2, wherein the biological samples are cells expressing the HSD17B7 gene, and in that step c) comprises measuring the expression of the said gene.

6. The in vitro method as defined by claim 4, in which the cells are sebocytes.

7. The in vitro method as defined by claim 4, in which the cells are cells transformed with a heterologous nucleic acid encoding hydroxysteroid (17-beta) dehydrogenase 7.

8. The in vitro method as defined by claim 2, in which the expression of the gene is determined by measuring the level of transcription of the said gene.

9. The in vitro method as defined by claim 2, in which the expression of the gene is determined by measuring the level of translation of the said gene.

10. The in vitro method as defined by claim 2, wherein step a) comprises preparing reaction mixtures comprising an enzyme hydroxysteroid (17-beta) dehydrogenase 7 and a substrate of the enzyme, and a reductase system, and in that step c) comprises measuring the enzyme activity.
